(19) United States
(12) Patent Application Publication
(10) Pub. No.: US 2018/0015282 A1
(43) Pub. Date: Jan. 18, 2018

(54) SYSTEM AND METHOD FOR TREATING SLEEP APNEA

(71) Applicant: Peerbridge Health, Inc., New York, NY (US)

(72) Inventors: Milton Waner, New York, NY (US); Peter D. Costantino, New York, NY (US)

(21) Appl. No.: 15/652,296

(22) Filed: Jul. 18, 2017

Related U.S. Application Data
(60) Provisional application No. 62/363,573, filed on Jul. 18, 2016.
(60) Provisional application No. 62/363,583, filed on Jul. 18, 2016.

(51) Int. Cl. A61N 1/36; A61N 1/372; A61N 1/378

(52) U.S. Cl. CPC A61N 1/3611; A61N 1/3787; A61N 1/3601; A61N 1/36139; A61N 1/37217

(57) ABSTRACT

The present disclosure is directed to a system for the treatment of a sleep disorder through stimulation of the hypoglossal nerve or the geniohyoid muscle of a patient, e.g. a human patient. In general, the system comprises three components, namely a sensing component 50, a stimulation component 100, and a control system 200. In some embodiments, the control system 200 may be embedded within the sensing component 50, or the control system 200 may be embedded within the stimulation component 100.

### CROSS-REFERENCE TO RELATED APPLICATIONS

This patent application claims priority to and the benefit of U.S. Provisional Application No. 62/363,573, filed Jul. 18, 2016; and also claims priority to and the benefit of U.S. Provisional Application No. 62/363,583, filed Jul. 18, 2016, the disclosures of each are hereby incorporated by reference herein in their entireties.

### BACKGROUND OF THE DISCLOSURE

Sleep apnea has been known for some time as a medical syndrome in two generally recognized forms. The first is central sleep apnea, which is associated with the failure of the body to automatically generate the neuro-muscular stimulation necessary to initiate and control a respiratory cycle at the proper time. Work associated with employing electrical stimulation to treat this condition is discussed in Glenn, “Diaphragm Pacing: Present Status”, Pace, V. I, pp 357-370 (July-September 1978).

The second sleep apnea syndrome is known as obstructive sleep apnea. Ordinarily, the contraction of the dilator muscles of the upper airways (nose and pharynx) allows their patency at the time of inspiration. In obstructive sleep apnea, the obstruction of the airways results in a disequilibrium between the forces which tend to their collapse (negative inspiratory transpharyngeal pressure gradient) and those which contribute to their opening (muscle contraction). The mechanisms which underlie the triggering of obstructive apnea include a reduction in the size of the superior airways, an increase in their compliance, and a reduction in the activity of the dilator muscles. The dilator muscles are intimately linked to the respiratory muscles and these muscles respond in a similar manner to a stimulation or a depression of the respiratory center. The ventilatory fluctuations observed during sleep (alternately hyper and hypo ventilation of periodic respiration) thus favor an instability of the superior airways and the occurrence of oropharyngeal obstruction. The respiratory activation of the genioglossus has been particularly noted to be ineffective during sleep. The cardiovascular consequences of apnea include disorders of cardiac rhythm (bradycardia, auriculoventricular block, ventricular extrasystoles, tachyarrhythmias) and hemodynamic (pulmonary and systemic hypertension). This results in a stimulatory effect on the autonomic nervous system. The electroencephalographic awakening is responsible for the fragmentation of sleep. The syndrome is therefore associated with an increased morbidity (the consequence of diurnal hypersomnolence and cardiovascular complications).

One method of treating obstructive sleep-apnea syndrome is to generate electrical signals to stimulate those nerves which activate the patient's upper airway muscles in order to maintain upper airway patency.

### BRIEF SUMMARY OF THE DISCLOSURE

In one aspect of the present disclosure is a sleep apnea treatment system comprising means for detecting an apnea event and means for stimulating a hypoglossal nerve or geniohyoid muscle in response to the detected apnea event, wherein the means for stimulating the hypoglossal nerve or geniohyoid muscle comprises a first portion which is subcutaneously implanted, and a second portion which is worn by the patient, the second portion configured to wirelessly transfer a stimulation pulse to the first portion, i.e. wirelessly deliver energy. In some embodiments, the means for detecting an apnea event include one or sensors for measuring a patient's vital signs, the one or more sensors being communicatively coupled to a control system. Applicants believe that the system described herein is minimally invasive, easy to use, and accurately provides stimulation therapy for the treatment of sleep apnea in a patient in need thereof.

In another aspect of the present disclosure is a system for the treatment of obstructive sleep apnea in a patient in need thereof, the system comprising a sensing component and a stimulation component, the sensing component comprising one or more wireless sensors for collecting or measuring one or more vital signs of the patient, the sensing component being in wireless communication with a control system, and wherein the stimulation component comprises (i) a surgically implantable body configured to deliver energy to one of a nerve or muscle; and (ii) a wearable appliance inductively coupled to the implanted body, the wearable portion configured to receive signals from the control system. In some embodiments, the vital signs are selected from the group consisting of blood oxygen, respiration rate, and heart rate. In some embodiments, the control system is embedded within the wearable apparatus of the stimulation component.

In some embodiments, the wearable portion is a dental appliance comprising a rechargeable battery, a pulse generator, and a means for inductively delivering energy to the surgically implantable body. In some embodiments, the means for inductively delivering energy is a transmitter coil. In some embodiments, the surgically implantable body comprises a receiver coil for receiving energy (i.e. a stimulation pulse) from the wearable portion. In some embodiments, the surgically implantable body is configured to deliver energy to a hypoglossal nerve. In some embodiments, the wearable portion further comprises components which enable wireless recharging.

In some embodiments, the wearable portion is a dermal device for positioning on the patient's skin, and wherein the dermal device includes a means for inductively delivering energy to the surgically implantable body. In some embodiments, the surgically implantable body is configured to deliver energy to a geniohyoid muscle. In some embodiments, the surgically implantable body comprises a means for wirelessly receiving energy from the dermal device, and wherein the surgically implantable device further comprises an insulating disc.

In another aspect of the present disclosure is an apparatus for treating sleep apnea comprising: an implantable body including a first member of a pair of inductive power transfer coils; and a wearable apparatus having a second member of the pair of inductive power transfer coils, a rechargeable battery, and a pulse generator; wherein the wearable apparatus is configured to wirelessly deliver energy to the implantable body upon receipt of a signal indicative of a sleep apnea event; and wherein the implantable body is configured to transfer the energy received from the wearable apparatus to a hypoglossal nerve or a geniohyoid muscle positioned in proximity thereto. In some embodiments, the wearable apparatus is a dental appliance adapted for placement over the patient's lower teeth. In some embodiments, the dental appliance is a bitesplint or retainer. In some embodiments, the wearable apparatus further comprises means for receiving control signals from a control system communicatively coupled thereto. In some embodiments, the control system comprises a processor, a memory, and a wireless communications module, the control system configured to (i) receive signals from one or more wireless sensors, (ii) process the signals to determine if a sleep apnea event has occurred or will occur, and (iii) send control signals to the wearable apparatus. In some embodiments, the control system is embedded within the dental appliance.

In another aspect of the present disclosure is a system for treating sleep apnea in a patient in need of treatment thereof comprising (i) one or more wireless sensors, (ii) a stimulation device, the stimulation device having a wearable portion and an implantable portion, the wearable portion configured to wirelessly transmit stimulation pulses to the implantable portion, and (iii) a control system, the control system having a memory coupled to one or more processors, the memory to store computer-executable instructions that, when executed by the one or more processors, cause the one or more processors to perform operations comprising (a) measuring vital signs of a patient using the one or more wireless sensors; (b) determining whether a sleep apnea event has occurred or will occur based on the measured vital signs; (c) facilitating the delivery of a stimulation pulse to treat sleep apnea using the stimulation component; wherein the control system is in wireless communication with both the one or more wireless sensors and the wearable portion of the stimulation component.

In some embodiments, the measured vital signs are used to derive a sleep apnea index and wherein the step of determining whether the sleep apnea event has occurred or will occur comprises comparing the derived sleep apnea index to a pre-determined sleep apnea index specific for the patient, the pre-determined sleep apnea index being stored in the memory. For example, if the system includes both a respiration sensor and a pulse oximetry sensor, and the processor computes an index or a weighted index of the two measured vital signs, stimulation therapy is provided when the derived index or derived weighted index falls below a pre-determined threshold index or a pre-determined threshold weighted index.

In some embodiments, the one or more wireless sensors include a respiration sensor, and wherein the step of determining whether the sleep apnea event has occurred or will occur comprises comparing measured respiration rates to a pre-determined threshold respiration rate. In some embodiments, stimulation therapy is administered when the measured respiration rate falls below the pre-determined threshold respiration rate.

In some embodiments, the one or more wireless sensors include a pulse oximetry sensor, and wherein the step of determining whether the sleep apnea event has occurred or will occur comprises comparing a measured blood oxygen content to a pre-determined threshold blood oxygen content. In some embodiments, stimulation therapy is administered when the measured blood oxygen content level falls below the pre-determined threshold blood oxygen content level.

In some embodiments, the system includes both a first sensor and a second sensor, each sensor monitoring a different vital sign of the patient, and stimulation therapy is applied when at least one of the sensors measures a vital sign level that falls below a pre-determined value specific for the patient. For example, if the first sensor is a respiration sensor and the second sensor is a pulse oximetry sensor, when at least one of a measured a respiration rate or a measured blood oxygen content level falls below a pre-determined respiration rate threshold or a pre-determined blood oxygen content threshold, stimulation is directed by the control system.

In some embodiments, the wearable apparatus is a dental appliance, and wherein the dental appliance comprises at least two transmission coils for delivering the stimulation pulses to two implantable bodies, the dental appliance adapted to releasably engage a portion of the patient's lower teeth, the at least two transmission coils positioned on an exterior surface of the dental appliance. In some embodiments, the control system is adapted to monitor the cessation of a sleep apnea event, e.g. when a measured/computer sleep apnea index returns to “normal” for the patient. In some embodiments, a stimulation pulse delivered does not exceed 10 seconds in duration. The skilled artisan will appreciate that the stimulation pulse frequency, amplitude, and rate may be determined on a per-patient basis such that a safe stimulus is provided to a nerve or muscle of the patient, but at the same time allowing for sufficient energy transfer to effectuate (at least temporarily) a cessation of a detected sleep apnea event.

In another aspect of the present disclosure is a computer-based system for monitoring and treating sleep apnea in a patient, the system comprising: one or more wireless sensors configured to monitor the patient for symptoms associated with sleep apnea; a stimulation component having a wearable apparatus and an implantable body; one or more processors; and memory comprising instructions executable by the one or more processors to cause the one or more processors to: (i) receive a set of physiological data from the one or more wireless sensors, (ii) detect, using a machine learning algorithm, an onset of a sleep apnea event based on the set of physiological data, and (iii) transmit a control signal to the wearable apparatus to cause the apparatus to wirelessly transmit power to the implantable body so as to stimulate a nerve or muscle of the patient. In some embodiments, the machine learning algorithm is a support vector machine, such as described in more detail herein.

In another aspect of the present disclosure is a computer-based system for monitoring and treating sleep apnea in a patient, the system comprising: one or more processors; and memory comprising instructions executable by the one or more processors to cause the one or more processors to: receive a set of physiological data from one or more wireless sensors, detect an onset of a sleep apnea event in response to the set of physiological data, and transmit a control signal to a stimulation component, the stimulation component including a dental appliance which wirelessly transfers a pulse of energy from the dental appliance to a surgically implanted electrode so as to stimulate the patient's hypoglossal nerve. In some embodiments, the dental appliance is a retainer adapted to position inductive energy transfer means in close proximity to a subcutaneously implanted body. The skilled artisan will appreciate that the dental appliance may be customized for each patient, and the location of the inductive energy transfer means of the dental appliance may depend upon where the implanted body, or a receiver coil thereof, is positioned during surgery. Of course, the dental appliance may be molded from an impression taken of the patient's lower teeth, and the various components of the dental appliance, as described herein, may be positioned within the dental appliance based on available space.

In another aspect of the present disclosure is a computer-based system for monitoring and treating sleep apnea in a patient, the system comprising: one or more processors; and memory comprising instructions executable by the one or more processors to cause the one or more processors to: receive a set of physiological data from one or more wireless sensors, detect an onset of a sleep apnea event in response to the set of physiological data, and transmit a control signal to a stimulation component, the stimulation component including a dermal appliance which wirelessly transfers a pulse of energy from the dermal appliance to a surgically implanted electrode so as to stimulate a geniohyoid muscle of the patient.

In another aspect of the present disclosure is a kit comprising: (a) a wireless controller; (b) one or more wireless sensors; and (c) a wireless stimulator, the stimulator having a wearable portion and an implantable portion, the wearable portion configured to wirelessly transfer a pulse of stimulation energy to the implantable portion. In some embodiments, the wireless controller is embedded within the one or more wireless sensors. In some embodiments, the wireless controller is embedded within the wearable portion of the stimulator. In some embodiments, the kit further comprises a recharger for recharging a power source of any of the aforementioned components.

### BRIEF DESCRIPTION OF THE FIGURES

FIGS. 1A-1C illustrate systems for treating sleep apnea.

FIGS. 2A and 2B illustrate systems for treating sleep apnea, and further illustrate the components of a stimulation component.

FIGS. 3A and 3B illustrate a dental appliance for wirelessly transferring energy to an implantable body.

FIGS. 4A-4D illustrate implantable bodies having various configurations.

FIG. 5 sets forth an overview of a computer system for treating sleep apnea.

FIG. 6 further illustrates a computer system for treating sleep apnea.

FIG. 7 provides a flowchart illustrating a method of treating sleep apnea.

FIG. 8 illustrates a charging device for recharging and/or cleaning a wearable apparatus or a sensing component.

FIG. 9 provides a flow chart illustrating the steps for charging a wearable component.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of. ” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Systems For Treating Sleep Apnea

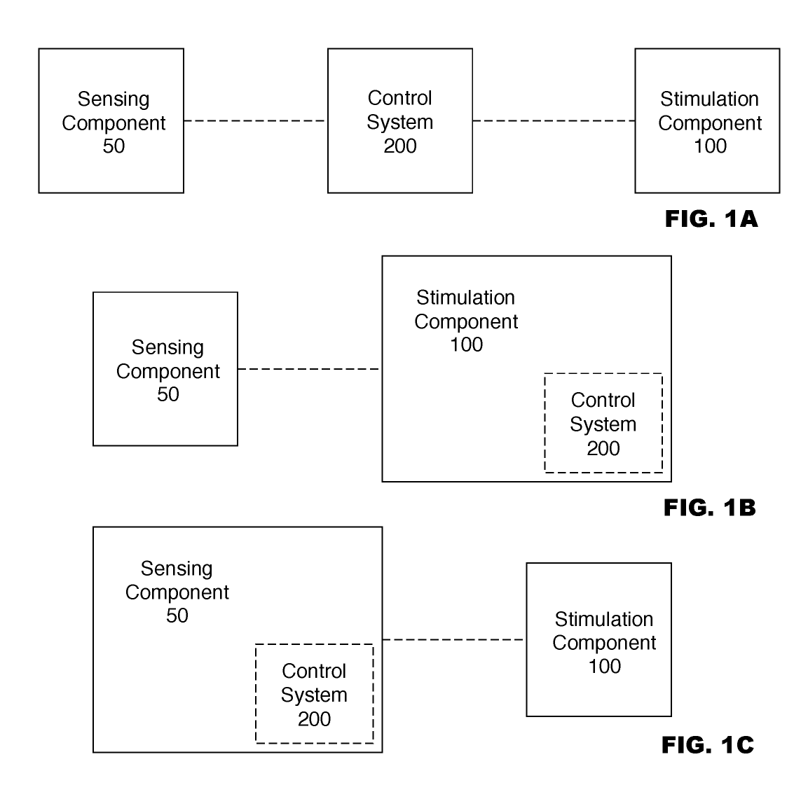

The present disclosure is directed to a system for the treatment of a sleep disorder through stimulation of the hypoglossal nerve or the geniohyoid muscle of a patient, e.g. a human patient. In general, and as depicted in FIG. 1A, the system of the present disclosure comprises three components, namely a sensing component 50, a stimulation component 100, and a control system 200. In some embodiments, and as depicted in FIGS. 1B and 1C, the control system 200 may be embedded within the sensing component 50, or the control system 200 may be embedded within the stimulation component 100.

The system for treating sleep apnea of the present disclosure is minimally invasive. Indeed, only a small implantable body 5 is surgically placed in proximity to the patient's hypoglossal nerve or geniohyoid muscle, and this small implantable 5 is acted upon by a wearable component 110, i.e. the wearable component 110 is configured to wirelessly deliver a stimulation pulse to the implantable body 5. Thus, in one aspect of the present disclosure is a system to provide a stimulation component having a wearable apparatus which wirelessly transfers energy to the small implantable body, such as through inductive charge coupling. In order to achieve the aforementioned, the stimulation component 100 of the present disclosure utilizes a pair of coils for inductive coupled power transfer. Inductive power coupling, as known in the art, allows energy to be transferred from a power supply (133) to an electric load (30) without connecting wires. In some embodiments, a power supply (133) is wired to a primary coil (120) and an oscillating electric potential is applied across the primary coil, thereby inducing an oscillating magnetic field. The oscillating magnetic field may induce an oscillating electrical current in a secondary coil (10 or 15) placed close to the primary coil (120). In this way, electrical energy may be transmitted from the primary coil (120) to the secondary coil (10 or 15) by electromagnetic induction without the two coils being conductively connected. When electrical energy is transferred from a primary coil (120) to a secondary coil (10 or 15) the coil pair are said to be inductively coupled. An electric load (30), here an electrode, wired in series with such a secondary coil (10 or 15) may draw energy from the power source wired to the primary coil (120) when the secondary coil (10 or 15) is inductively coupled thereto. Each of the components of the system are described in more detail herein.

Sensing Component

The systems for treating sleep apnea disclosed herein comprise a sensing component 50 having one or more wireless sensors for measuring physiological data, e.g. a patient's vital signs. The physiological data can be related to one or more of the patient's sleep patterns, sleep apnea events, normal physiological events, and/or abnormal physiological events. In various embodiments, the sensors monitor physiological data from a patient in real-time, and can transmit this sensor data as one or more signals to be received by the control system 120, as described herein. The wireless sensor data can be indicative of events and/or patient symptoms, such as symptoms associated with the onset of a sleep apnea event (such that stimulation with the stimulation component 100 may be initiated), and/or a lessening of symptoms associated with a sleep apnea event (such that stimulation may be terminated). In some embodiments, the sensor data is indicative of sleep patterns of the patient and/or physiological information of the patient during sleep.

Wireless sensors utilized in the systems of the present disclosure may monitor or track any one of a variety of patient symptoms or status indicators, including sounds, such as snoring, breathing, cessation of breathing, heartbeat, patient position, and the like. In some embodiments, the sensors can monitor breathing patterns, including changes in the pace of breathing, length between breaths, lengths of inhalation, and the like. Other patient symptoms to be sensed can include temperature, temperature changes, and the like. Other data that may not be directly related to sleep apnea can also be measured, including saliva, its content, or other markers. Physiological information that can be monitored by the sensors described herein includes, without limitation, breathing sounds, snoring sounds, breathing rate, respiratory air flow, respiratory rate, chest expansion, blood oxygen level, cardiac data (e.g., heart rate, EKG data), sleeping position, sleeping movements, blood pressure, brain activity (e.g., EEG data) and/or variants thereof and/or combinations thereof

Any suitable number and combination of wireless sensor types may be used, such as one, two, three, four, five, or more different sensor types. Exemplary sensor types suitable for incorporation with embodiment herein include, but are not limited, to audio sensors (e.g., microphones), video sensors (e.g., cameras), blood oxygen sensors (e.g., pulse oximeters), air flow sensors, motion sensors, temperature sensors, strain gauges, force sensors, pressure sensors, heart rate monitors, blood pressure monitors, EKG sensors, EEG sensors, or any other sensor type suitable for obtaining physiological information relating to the patient's sleep status and/or sleep apnea status.

A single wireless sensor may include different sensing components for monitoring a plurality of different vital signs. For example, one wireless sensor can include a pressure detector for monitoring the pulse rate, and also can include an electrochemical detector for blood glucose level measurement (the glucose level can also be measured by an infrared detector or eye scanner). By way of another example, a wireless sensor can include a surface-attached sensing component, such as one or more ECG electrodes, and can include an implantable sensing component, such as an implanted intracardiac pressure transducer coupled to a heart chamber (e.g., the right ventricle). The skilled artisan will appreciate that different wireless sensors of different types for monitoring different vital signs can be conveniently worn by or implanted in the patient depending on the needs of care for the patient.

The wireless sensors as described herein may be surface-attachable sensors suitable for attachment to the skin of a subject, or implantable sensors suitable to be implanted in the body of the subject. In some embodiments, the one or more wireless sensors are configured to detect a signal corresponding to a physiological condition, such as vital signs or other signs of interest, including hemodynamic parameters of a patient, neuromuscular signals or the like. By way of a specific example, hemodynamics, as known in the art, relates to the study of blood flow. The circulatory system, including the heart, the arteries, the microcirculation, and the vein, functions to transport the blood to deliver O2, nutrients and chemicals to the cells of the body, and to remove the cellular waste products. The heart is the driver of the circulatory system generating cardiac output (CO) by rhythmically contracting and relaxing. This creates changes in regional pressures, and, combined with a complex valvular system in the heart and the veins, ensures that the blood moves around the circulatory system in one direction. Hemodynamic parameters (or properties), as described herein, include the physiological conditions associated with the blood flow, which includes not only the physical characteristics of the blood flow itself, e.g., blood flow rate, blood flow pressure, and pulse rate, but also those parameters relating to the blood components such as cells, proteins, chemicals, etc.

In some embodiments, the sensing component 50 includes a respiration sensor. A respiration sensor detects, either directly or indirectly, whether the subject is breathing to detect apnea an apnea event. The respiration sensor produces a sensor signal that includes cyclic variations indicative of inhaling and exhaling. For example, a thoracic impedance sensor includes cyclic variations as the subject inhales or exhales. In certain other examples, blood pressure and heart sound signals include components that are indicative of cyclic variations as the subject inhales or exhales. When so configured, a blood pressure sensor or a heart sound sensor may also be considered a respiration sensor.

In some embodiments, the sensing component includes one of a blood pressure sensor or a heart sound sensor (e.g. a non-respiration-based sensor which measures a vital sign parameter indicative of apnea other than whether the subject is breathing). For example, certain other components of blood pressure and heart sound signals do not include the cyclic variations resulting from inhaling and exhaling. For wireless sensors that are configured to detect ECG signals, the sensors can be attached to the skin of a patient for ECG signals recordation in a manner that is similar to the configuration of traditional 3-lead, 5-lead, or 12-lead ECG leads. In certain embodiments, the wireless sensors can be arranged in one or more groups of electrodes each arranged in, for example, an orthogonal configuration.

In some embodiments, the sensing component 50 includes a wireless sleep monitor. The wireless sleep monitor can include one or more antennas, with each of the one or more antennas configured to receive electromagnetic radiation and/or transmit electromagnetic radiation, and may be configured to measure chest movements i.e. inhalation and exhalation.

Wireless sensors can be deployed on a patient for monitoring sleep apnea, including one or more of an accelerometer to detect movement of the chest, an ECG sensor or sensors to obtain information about the patient's heart rhythm, and an oxygen saturation sensor worn, for example, on a patient's finger. Alternatively, or in combination, the use of hybrid sensors can provide more comprehensive information regarding the patient's condition in a more efficient and/or more reliable manner. For example, monitoring different vital signs simultaneously using different types of wireless sensors can provide redundancy and improved robustness of monitoring quality as well as facilitate reconciliation of inconsistencies among the data gathered from different types of sensors (for different vital signs), reduce false alarm rates, etc. The skilled artisan will appreciate that if a plurality of vital signs are collected, the data may be index or a weight index may be generated, and that index or weighted index may be used by the computer systems, described further herein, to determine whether a sleep apnea event has occurred or is likely to occur (e.g. by comparing a computer or derived index or weighted index to a pre-determined threshold index or a pre-determined threshold weighted index, each specific to the particular patient being treated).

More than one wireless sensor can form a network, e.g., a mesh network. Each of the sensors can include a sensing component configured to detect a signal corresponding to at least one physiological condition or vital sign of the patient, and a communication component configured to wirelessly transmit the detected signal to either another wireless sensor or to the control system 200. The sensing component 50 or the individual wireless sensors thereof may include a rechargeable battery, and the battery may be recharged either wireless (inductive coupling as described herein and thus comprises an appropriate receiver coil) or may be charged in a more traditional wired manner. The communication component of selected sensors can also be configured to receive and/or relay signals transmitted from other wireless sensors on or in the body.

The wireless sensors or the network of wireless sensors can continuously monitor selected vital signs of the subject, and communicate the signals acquired from the sensing components via the communicating components of the sensors to the control system 120. Each of the wireless sensors can be programmed such that signals detected by the sensor falling into a predetermined (e.g., an acceptable or normal) range are not transmitted, or transmitted at a lower frequency. The acceptable range for signals for different patients and for each wireless sensor can be set individually, for example, based on the type of the sensor, the patient's condition, the therapy being used by the patient, etc. As described herein, the control system 120 can include a communication component configured to wirelessly receive signals from each of the plurality of wireless sensors, and send data and/or command to each of the plurality of wireless sensors. The control or master node can further include a monitoring unit coupled with the communication component. For example, the monitoring unit can include a readable medium and a processor coupled to the computer readable medium. The computer readable medium can store coded instructions for execution by the computer processor, which, upon the execution of the instructions, carries out pre-designed tasks, such as a classification task or sleep apnea detection task.

In a system where there is more than one wireless sensor, all of the wireless sensors can each individually transmit the collected physiological data to the control system 120. Alternatively, one of the wireless sensors can include hardware and software configured to serve as a master node or gateway that receives detected physiological data from other wireless sensors, and forward such signals via a radio (e.g., WiFi) link to the control system 120 at an appropriate rate (e.g., to save battery power of the sensors). The transmitted physiological data can be processed by the control system 120 with an appropriate program or set of instructions.

Other components of wireless sensors, including methods of detecting vital signs and/or transmitting signals to a control system 120 are described in U.S. Pat. Nos. 7,979,111 and 9,101,264, the disclosures of which are hereby incorporated by reference herein in their entireties.

Stimulation Component

As noted herein, the system for treating sleep apnea also comprises a stimulation component 100. The stimulation component 100 itself comprises two discrete portions, namely (ii) an implantable body 5, configured for surgical implantation within the patient; and (ii) a wearable apparatus or appliance 110 configured to be “worn” by the patient, such as in contact with a skin of the patient (a “dermal” appliance) or within the patient's mouth (a “dental” appliance). Each of these discrete portions of the stimulation component 100 will be described in more detail herein.

Wearable Apparatus

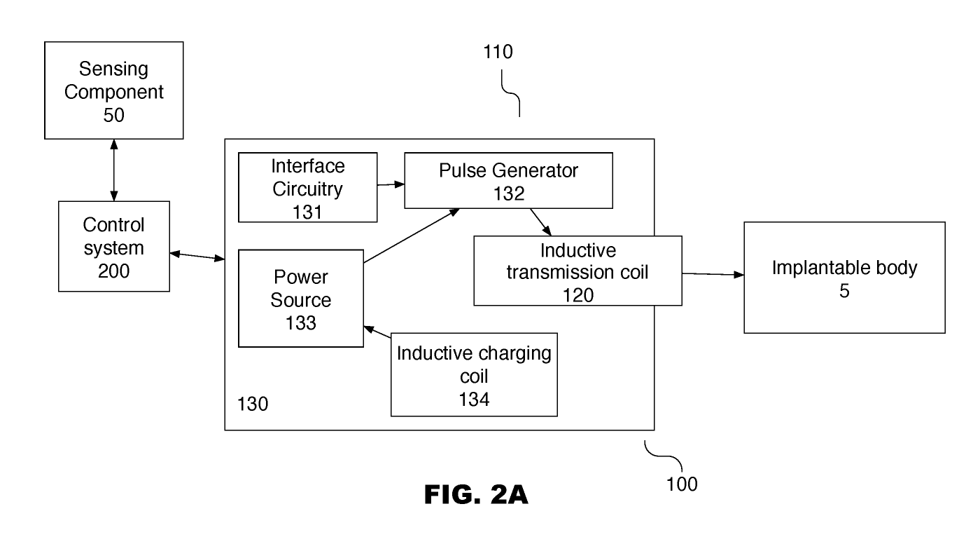

With reference to FIG. 2A, the wearable apparatus 110 includes a housing 130 including interface circuitry 131 for receiving and/or processing signals from the control system 200, a pulse generator 132 for generating stimulation pulses, a power source 133 (e.g. a rechargeable lithium-ion or nickel-cadmium battery), and inductive power coupling means 120 for wirelessly transmitting energy to the surgically implantable body 5. With reference to FIG. 2B, the skilled artisan will appreciate that in embodiments where the control system 200 is embedded within the wearable apparatus 110, the control system 200 assumes the role of interface circuitry 131, i.e. the control system 200 provides signals to the pulse generator 132 to effect stimulation of the hypoglossal nerve or geniohyoid muscle of a patient upon determination that a sleep apnea event has occurred or will occur. In some embodiments, the wearable apparatus 110 further includes a recharging receiver coil 134 designed to receive energy to recharge the power source 133 (as will be further described herein).

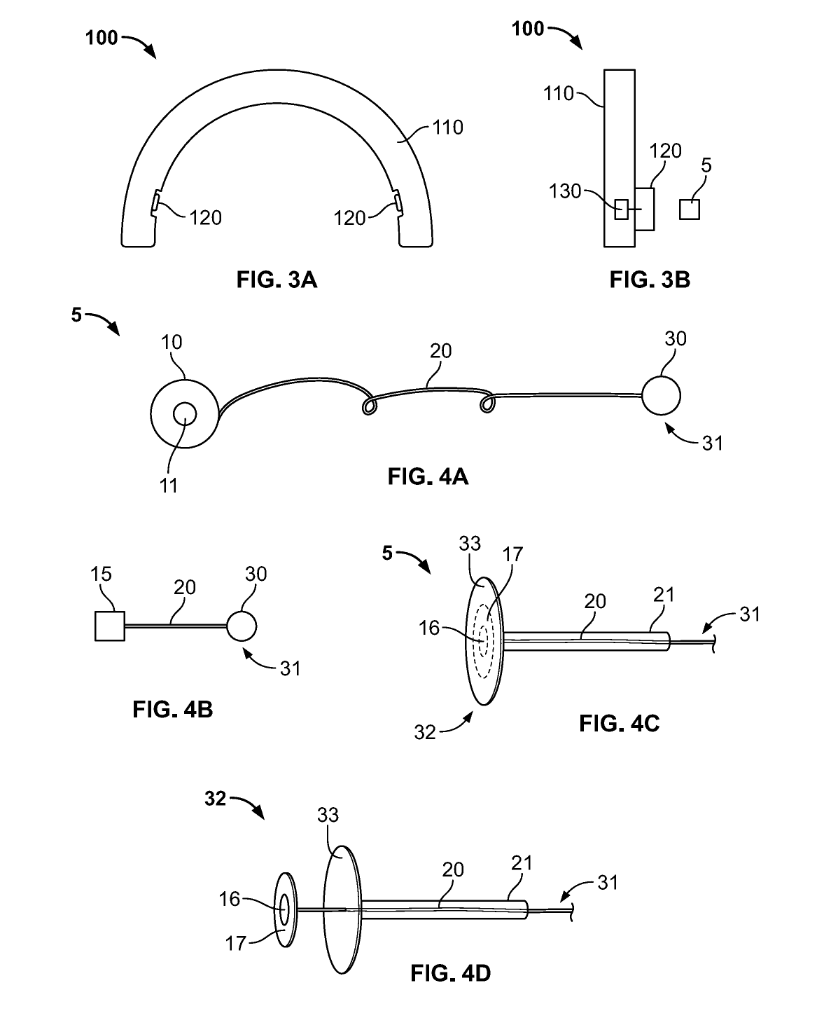

As used herein, the terms “inductive coil,” “inductive transfer coil,” or the like refer to a coil that is used to receive and/or transmit inductive energy wirelessly. Such inductive energy transmission may be realized by regular inductive coupling or by exploiting magnetic resonance. The inductive power coupling consists of a first inductive coil 120 and a second inductive coil 10 or 15 (see also FIG. 4A and 4B). The first coil 120 is wired to the power supply 133 through the pulse generator 132 to drive the first coil 120. The pulse generator may include a switching unit providing a high frequency oscillating voltage supply, for example. The skilled artisan will appreciate that when the secondary coil 10 or 15 is brought into proximity with the primary coil 120, the pair of coils forms an inductive couple and power is transferred from the primary coil 120 to the secondary coil 10 or 15.

In some embodiments, the inductive power coupling means 120 is a first member of a pair of inductive coils. In some embodiments, the inductive power coupling means 120 for wireless transmitting energy to the surgically implantable body 5 is an inductive power transfer coil or a transmitter coil. In some embodiments, the receiver coil 10 or 15 of the implantable body 5 is provided in electrical communication with the transmitter coil 120 of the wearable apparatus 110 for receiving power or energy when suitable aligned.

In some embodiments, the receiver 10 or 15 and transmitter 120 coils may be formed from a wire or other suitable conductive element that may be configured, for example, to form a plurality of concentric loops or converging, spiraling circles. In some embodiments, wire forming the receiver and/or transmitter coils is formed from a suitable conductive material including, but not limited to, metals, conductive polymers, conductive composites and the like. It is understood that the receiver 10 or 15 and transmitter 120 coils may be formed from any suitable material and may be configured in a variety of geometries to allow the transfer of power from the wearable apparatus and to the implantable body. Further, the size, shape, spacing and/or location of receive inductive coil 120 and constituent loops may vary between embodiments.

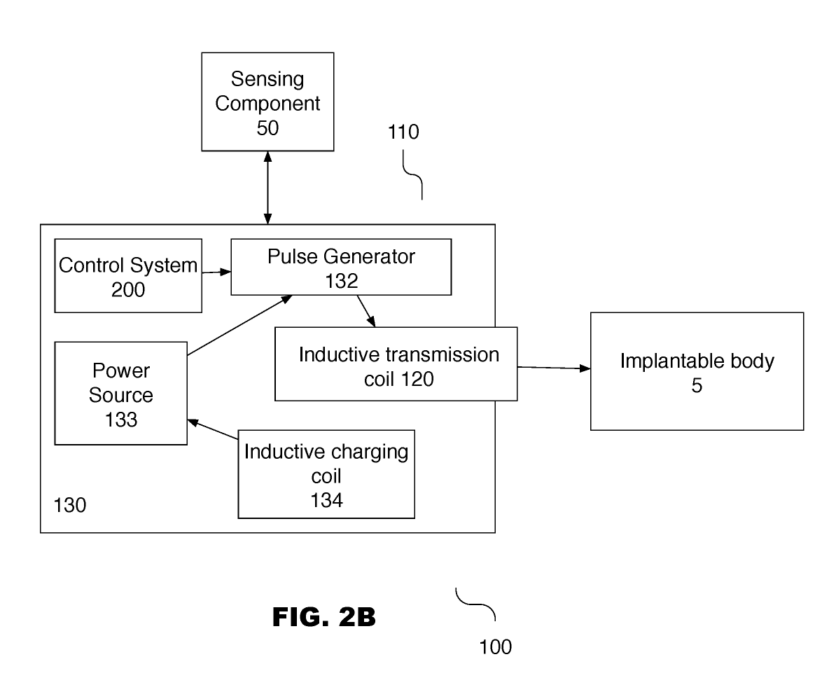

In some embodiments, the wearable apparatus 110 is a dental appliance such as depicted in FIG. 3A. In some embodiments, the dental appliance resembles an orthodontic retainer which is oriented to the teeth of the lower jaw. In some embodiments, the dental appliance covers all of the teeth of the lower jaw, and comprises a U-shaped base. In other embodiments, less than the entire set of teeth are covered by the appliance. The skilled artisan will appreciate that the dental appliance may be configured to intimately and releasably engage with at least one tooth of the lower jaw, thus securing the dental appliance in position. The skilled artisan will also appreciate that the size and shape of the dental appliance may be adapted to encompass each of the components of wearable device 110 noted herein.

As shown in FIG. 3A, the dental appliance includes two transmitter coils 120 to wirelessly transferring energy to two implantable bodies 5, where each implantable body is surgically implanted proximal to a hypoglossal nerve. The skilled artisan will appreciate that the transmitter coils 120 may be placed at any position along the dental appliance, the positions being determined by where in the mouth the receiver coils 10 or 15 of the implantable body are surgically placed. FIG. 3B illustrates a profile view of the stimulation component 100 comprising a dental appliance 110 and an implantable body. In some embodiments, the transmitter coils 120 are positioned external to the dental appliance.

In some embodiments, a dermal appliance includes a transmitter coil 120 to wirelessly transfer energy to an implantable body. In some embodiments, the dermal appliance may also comprise a magnet having a first polarity such that the dermal body may be positioned over and coupled to a portion of an implantable body including a magnet 17 having a second polarity. In some embodiments, the magnet of the dermal appliance is embedded within or integral with the transmitter coil 120.

Surgically Implantable Body

With reference to FIGS. 4A and 4B, in some embodiments the surgically implantable body 5 comprises inductive power coupling means 10 or 15 and an optional electrode 30 for stimulating a hypoglossal nerve. In some embodiments, the inductive power coupling means 10 or 15 is a second member of a pair of inductive coils. In some embodiments, the inductive power coupling means 10 or 15 is a receiver coil for wirelessly receiving energy via inductive charge coupling from the wearable apparatus 110. In some embodiments, the inductive power coupling means 10 is in the form of an eyelet having an opening 11 in which a screw, e.g. a self-tapping screw, may be inserted such that the implantable body 5 may be secured to the patient's mandible.

In some embodiments, the implantable body 5 comprises an insulated wire or lead 20 such that the electrode 30 may be positioned at a distance from the means for receiving energy 10 or 15. In some embodiments, a sheath 21 (e.g., a biocompatible polymer) is used to insulate the wire 20 along its length except for the distal end 31 and proximal end 32. In some embodiments, the electrode 30 comprises a barb or a hook for positioning proximal the hypoglossal nerve. In other embodiments, the electrode comprises a cuff for positioning at least partially around the hypoglossal nerve.

In operation, an electrical stimulus is delivered by the wearable apparatus 110 to the receiver coil 10 or 15 and through the stimulation wire/lead 20 to the electrode 30 proximal a nerve innervating a muscle controlling upper airway patency to mitigate obstruction thereof, as in FIGS. 4A and 4B.

In some embodiments, the implantable body 5 is implanted in a patient and disposed in a subcutaneous pocket, whereby the electrode is disposed proximal to a hypoglossal nerve to innervate the genioglossus muscle. In some embodiments, the wire/lead 20 is disposed in a subcutaneous tunnel. In some embodiments, the electrode 30 may be attached to a specific branch of the hypoglossal nerve innervating the genioglossus muscle, or may be attached to a more proximal portion (e.g., trunk) of the hypoglossal nerve. Without wishing to be bound by any particular theory, it is believed that activating the genioglossus muscle causes the tongue to protrude thus increasing the size of anterior aspect of the upper airway or otherwise resisting collapse during inspiration.

With reference to FIG. 4C, in some embodiments the implantable body 5 comprises an insulated wire/lead 20, whereby the wire/lead 20 is insulated in a biocompatible polymer 21 along its length, except for the proximal end 32 and distal end 31. In some embodiments, the implantable body comprises a means 16 for wirelessly receiving energy via inductive charge coupling from the wearable apparatus 110 (e.g. a receiver coil). In some embodiments, the implantable body further comprises an insulating disk 33 located at the proximal end of the wire 32. In some embodiments, the means for wirelessly receiving energy 16 is integrated within disk 33 (FIG. 4C); or the means 16 may be spaced apart from the disk (FIG. 4D). For example, while the disk 33 may be comprised of an insulating material, embedded within the disk 33 may be a receiver coil 16 configured to receive a wireless transmission of energy. In some embodiments, the implantable body further comprises a magnet 17 such that a dermal appliance 110 having a magnet may releasably engage the magnet 17. In some embodiments, the magnet may be embedded within the disk 33, or may be part of the receiver coil 16.

In some embodiments, the insulating disk 33 has a diameter ranging from about 0.25 cm to about 3 cm. In other embodiments, the insulating disk 33 has a diameter ranging from about 0.55 cm to about 2.5 cm. In yet other embodiments, the insulating disk 33 has a diameter ranging from about lcm to about 2 cm. In some embodiments, the insulating disk 33 is adapted to prevent electrical stimulation of the nervous system or muscles around the proximal end 32 of the wire that is disposed in the neck of a patient. In operation, an electrical stimulus may be delivered by the wearable apparatus 110 to a receiving coil 15 and through the stimulation wire/lead 20 to its unsheathed distal end 31 to stimulate the geniohyoid muscle.

In some embodiments, the implantable body 5 may be introduced transcutaneously from the platysma muscle through the skin under the neck. For optimal stimulation efficiency and patient comfort, in some embodiments the distal end of the wire 31 is positioned sufficiently close to the hypoglossal nerve or geniohyoid muscle so as to provide good stimulation with low electric current, but not touching the nerve itself.

In some embodiments, the insulating disk 33 can be expandable so that it is easier to deploy subcutaneously, e.g., by way of a catheter. Then, the skin is sutured or otherwise closed, with the disk 33 slightly below and parallel to the skin of the neck. After closure, the skin that covers the embedded proximal end of the wire 32 can be marked with ink or other suitable marker. A wearable component 110 can be provided in the form of a pad or disk that can be adhered to the skin over the proximal end of the subcutaneous component 50, and positioning of this pad 110 by the patient can be facilitated by the mark on the skin.

To verify the correct positioning of the implantable body 5 and/or that the distal end 31 is advanced to a suitable location, a calibration can be performed using a sensor to detect the movement of the tongue in response to stimulation pulses (e.g., in some embodiments between about 1 Hz and about 20-200 mA current; and in other embodiments between about 20 to about 30 mA). In some embodiments, the calibration method measures electro myographic tongue movements, which are measurable, such as visually (e.g. looking for a tongue twitch). In other embodiments, calibration may be performed using ultrasonographic measurement or high speed photography to measure tongue movements.

Control System

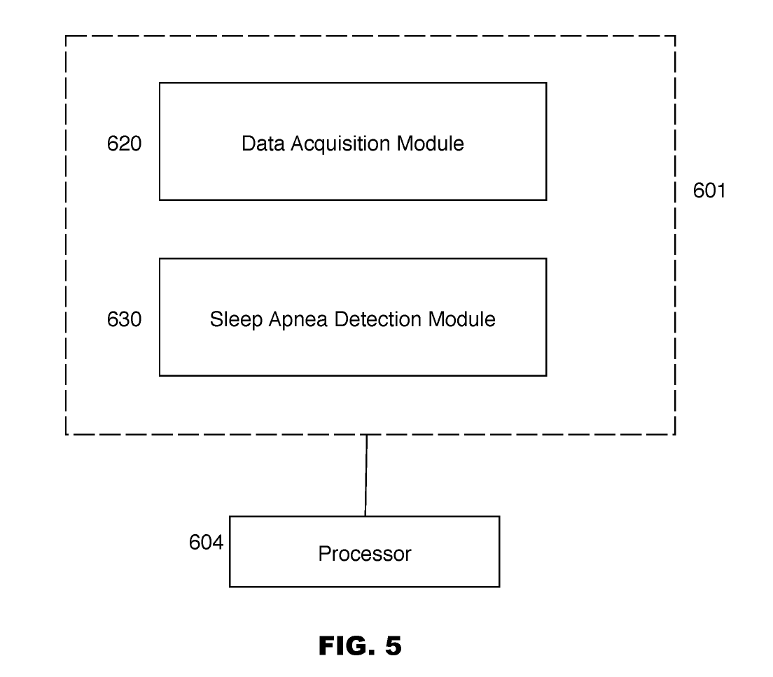

FIG. 5 provides an overview of the various modules utilized within the presently disclosed sleep apnea treatment system. In some embodiments, the sleep apnea treatment system employs a computer device having one or more processors 604 and at least one memory 601, the at least one memory 601 storing non-transitory computer-readable instructions for execution by the one or more processors 604 to cause the one or more processors 604 to execute instructions in one or more modules. In some embodiments, a non-transitory computer-readable media may comprise all computer-readable media except for a transitory, propagating signal. In some embodiments, the present disclosure provides a computer-implemented method comprising the steps of (a) running a physiological data acquisition module 620 to receive vital signs from the patient from one or more wireless sensors; and (b) running a sleep apnea detection module 630 to process the acquired physiological data and determine whether a sleep apnea event has occurred or likely will occur. The skilled artisan will also appreciate that additional modules may be incorporated into the workflow. In some embodiments, the system is configured to monitor and/or measure the patient's physiological characteristics and/or sleep status using module 620, and then determine whether a sleep apnea event has occurred or will imminently occur using module 630. If the onset of a sleep apnea event is detected using module 630, the system for treating sleep apnea 600 can control the stimulation component 100, which may wirelessly transfer energy to an electrode 30 surgically implanted proximal a hypoglossal nerve or geniohyoid muscle of a patient.

In some embodiments, the sleep apnea detection module 630 includes, for example, instructions for pattern recognition to recognize a potential sleep apnea onset based upon incoming data from the sensor(s). Examples of such instructions for sleep apnea detection is described in United States Patent Publication No. 2014/0180036, which is entitled “Device and Method for Predicting and Preventing Obstructive Sleep Apnea Episodes,” the disclosure of which is hereby incorporated by reference herein in its entirety. Both detection and training can be concurrent, for example, so that the monitoring unit “learns” the specifics of the patient to more accurately predict future sleep apnea events as noted herein (see discussion of machine learning).

In other embodiments, the sleep apnea detection module 630 includes algorithms for comparing measured values to pre-determined threshold values. In yet other embodiments, the sleep apnea detection module 630 includes algorithms to create an index of two or more values, such as an index or weighted index derived from first and second sensors, each sensor monitoring or measuring a separate vital sign. Once the sleep apnea detection module 630 computes the index, the index may be compared to a pre-determined index value. The algorithms and any collected data may be stored in a memory of the system.

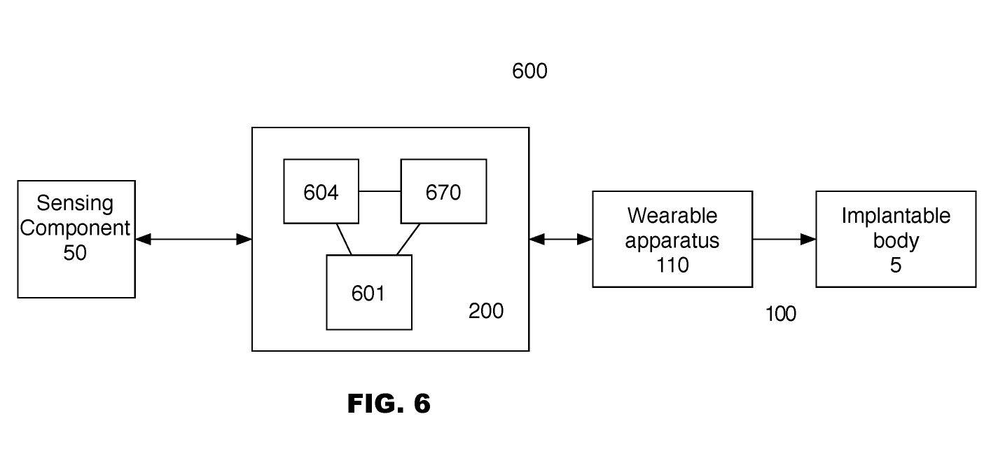

FIG. 6 illustrates an exemplary computer-based system 600, comprising a sensing component 50 including one or more wireless sensors (e.g., n sensors, where n is one, two, three, four, five, or more), a processor 604, a communication circuitry 670, and a stimulation component 100 comprising a wearable apparatus 110 and an implantable component 5. In various embodiments, processor 604 can comprise a single processor or a plurality of processors. System 600 can further comprise a memory 601 having executable instructions stored thereon, and the processor 604 can be configured to execute the instructions stored on the memory device. Wireless sensors can be configured to monitor a patient for sleep apnea symptoms. In some embodiments, the processor 604 and one or more of wireless sensors can be part of an integrated system housed in a single housing component.

Processor 604 can execute instructions to receive physiological data from wireless sensors, and to detect, identify, and/or assess sleep apnea events based on or in response to the physiological data or vital signs. Processor 604 can also execute instructions to transmit control signals to the stimulation component 100. Processor 604 can perform any one or more of the functions ascribed to it herein by executing one or more algorithms, including but not limited to machine learning algorithms, as described further herein.

Computer-based systems of the present disclosure provide one or more processors 604 that can receive sensor data and use the sensor data to detect, predict, and/or assess a patient's symptoms, such as symptoms associated with sleep apnea. For example, in some embodiments, processor 604 can execute instructions to detect physiological events such as onset or termination of a sleep apnea event. In some embodiments, processor 604 can execute instructions to identify physiological discrepancies such as a discrepancy between a current sleeping pattern and a previous sleeping pattern, such as a measured pattern stored in memory 601. In some embodiments, processor 604 can execute instructions to identify physiological discrepancies such as a discrepancy between a measured sleep apnea index and a previously determined sleep apnea index (such as a sleep apnea index determined using measured data from a prior night's sleep, or a sleep apnea index determined in a clinical setting). In some embodiments, processor 604 can execute instructions to make physiological assessments such as an assessment of the likelihood that an apnea event will begin or terminate.

In some embodiments, processor 604 can execute instructions to identify differences between a derived or computed sleep apnea index and a pre-determined sleep apnea index specific for a particular patient (such as one pre-determined for a patient in a sleep center). The conventional diagnosis of obstructive sleep apnea (OSA) relies on testing done during an overnight sleep study using polysomnography. A value referred to as the apnea hypopnea index (AHI) is the average number of apneas and hypopneas per hour of sleep determined from the polysomnographic study. The AHI index values have been used to classify OSA as mild (AHI=5-15), moderate (AHI=15-30), and severe (AHI>30). While apnea is defined as the cessation of airflow for more than 10 seconds, the definition of hypopnea is yet to be standardized. In addition to the original (Chicago) definition of hypopnea that requires either >50% airflow reduction or a lesser airflow reduction with associated >3% oxygen desaturation or arousal, two other stricter definitions have been used by others. In some embodiments, the processor 604 can derive a sleep apnea index (e.g. using the sleep apnea determination module 630) using data collected from the sensing component 50. The newly derived sleep apnea index may then be compared to the patient's clinically derived sleep apnea index and stimulation provided if the newly derived AHI exceeds the clinically derived AHI (or, for that matter, some pre-determined threshold AHI specific for the patient).

In some embodiments, processor 604 can execute instructions to measure a vital sign, or a combination of vital signs, and determine whether the vital sign or the combination of vital signs meets or exceeds a pre-determined threshold value specific for the patient.

The control system 120 may be able identify, with aid of the one or more processors and using collected data from the wireless sensors and/or data sorted in a memory 601, a discrepancy or difference between measured sleeping patterns of the patient and previously derived sleep patterns (e.g. sleeping patterns determined in a clinical setting; or sleeping patterns determined using the system 600 and stored in memory 601); and classifying the discrepancy as a sleep apnea event.

In some embodiments, the systems 600 herein include one or more processors 604 that can automatically collect and analyze some or all of the patient parameters which have been sensed by one or more wireless sensors. By tracking these parameters, and identifying changes in these patterns overtime, certain patient parameters and/or symptoms may be correlated with the onset of sleep apnea, snoring, or the like. In those embodiments, these identified patient parameters may be then relied on to help predict the onset of an apnea event in order to begin treatment, i.e. stimulating the hypoglossal nerve or geniohyoid muscle. In some embodiments, the processor is configured to implement a machine learning algorithm that identifies patient-specific correlations between physiological parameters and/or symptoms and sleep apnea events, and uses these patient-specific correlations to predict the onset of a sleep apnea event. Symptoms that may be correlated with onset of a sleep apnea event include but are not limited to: changes in blood oxygen level, changes in heart rate, changes in breathing rate or rhythm, changes in body temperature, changes in electrical resistance (e.g., of the skin), increase in sweating, or decrease in sweating. Computer-based approaches such as machine learning algorithms can be used to determine combinations of physiological parameters and/or symptoms that are useful for detecting the onset of sleep apnea events.

In some embodiments, the control system 120 is capable of receiving data from both the sensing component 50 and from the stimulation component 100. For example, the stimulation component 100 may record the parameters associated with given stimulation pulses over time, and this data, along with physiological data collected from the sensing component 50, may be used by a clinician to titrate the system or to otherwise analyze a sleep state of the patient. The collected data may also be used to generate patient alarms and reports on patient treatment and status. The collected data may also be stored in memory 601 or wirelessly communicated to a clinician. For example, the collected data may be used to identify irregularities in the sleep patterns and if appropriate take action, e.g., send an alert for help. The data collected over time can be useful to identify problems early on, e.g., worsening breathing patterns, worsening sleeping problems, etc. The data can then be considered by the treating healthcare professional and/or automatically assessed by the processor. Likewise, by collecting data from an individual patient over time, the system can “learn” patient specific patterns of sleep and patient specific patterns of apnea, e.g., via machine learning algorithms, which can enable the system to predict when an event is likely to occur and enable the system to calibrate and select to what level to activate the device.

Computer-based systems of the present disclosure provide one or more processors 604 that can transmit a control signal to the stimulation component 100. For example, in some embodiments, processor 604 can execute instructions to detect, predict or assess a pre-apnea or apnea event based on or in response to received physiological data from wireless sensors, and can execute instructions to transmit a control signal to the stimulation component 100 and the stimulation component 100, when in use, to wirelessly transfer energy from a wearable apparatus 110 to the surgically implantable apparatus 5 to stimulate the hypoglossal nerve or the geniohyoid muscle of the patient. In some embodiments, when the wearable apparatus 110 is a dental appliance positioned in the patient's mouth, processor 604 can send a control signal to dental appliance to cause it to transfer energy to the implantable component 5.

Methods of Treating Sleep Apnea

The present disclosure also provides a method of stimulating a hypoglossal nerve or a geniohyoid muscle of a patient. In some embodiments, the method includes attaching at least one electrode in proximity to the patient's hypoglossal nerve and applying an electric signal through the electrode to at least one targeted motor efferent located within the hypoglossal nerve to stimulate at least one muscle of the tongue. In other embodiments, the method includes attaching at least one wire/lead in proximity to the patient's geniohyoid muscle and applying an electric signal through the wire/lead.

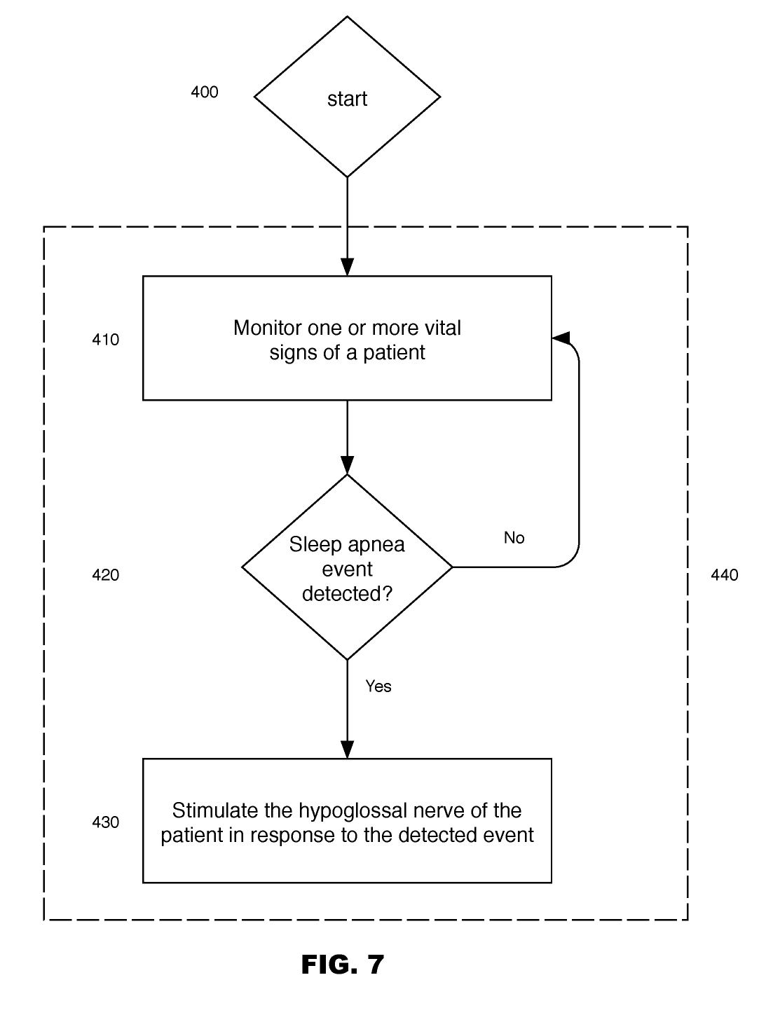

FIG. 7 illustrates a method for detecting and treating a sleep apnea event, in accordance with embodiments. This method, as with all other methods described herein, can be performed by any embodiment of the systems, devices, and apparatus provided herein. For example, one or more steps of the method can be performed by one or more processors of a system (e.g., processor 604 of the system 600) for monitoring and treating a patient's sleep apnea. In some embodiments, the method comprises applying an electric signal through at least one electrode to a hypoglossal nerve to stimulate at least one muscle of the patient's tongue. In other embodiments, the method comprises applying an electric signal through at least one wire/lead to the patient's geniohyoid muscle. In some embodiments, the system may be configured to determine in real-time or near real-time the occurrence of a sleep apnea event in a patient in need of treatment thereof (e.g. using the one or more sensors and a control system), and send a signal to the stimulation component to deliver an appropriate electrical stimulation therapy to the hypoglossal nerve or geniohyoid muscle of the patient during the apnea event to treat the apnea event.

In some embodiments, for example, the physiological data may be collected (step 410) from the patient while the patient is asleep. Each sensor can provide respective sensor data (e.g., to the controlling processor) throughout the monitoring period at predetermined time intervals, or continuously. The rate at which sensor data is provided can be varied as desired to ensure accurate monitoring of the patient's sleep status and/or sleep apnea status. For example, the vital signs of the patient may be monitored with the one or more sensors of the sensing component 50 every second, every five seconds, every ten seconds, etc. If no sleep apnea event is detected (step 420), the physiological measurements may continue to be collected and analyzed, but no stimulation is provided via the stimulation component 100 (i.e. the sleep apnea detection module 630 has not identified a sleep apnea event, and monitoring for such an event continues).

In some embodiments, an onset of a sleep apnea event is detected based on or in response to the set of sensor data. The sensor data can be indicative of physiological parameters and/or symptoms of the patient that are associated with the onset of the sleep apnea event. For instance, the sensor data can indicate that the sleep apnea event has occurred and/or is occurring. Alternatively, or in combination, the sensor data can indicate that a sleep apnea event is about to occur and/or is likely to occur. In some embodiments, the step 420 is performed using a computer-implemented algorithm (module 630), which may or may not be a machine learning algorithm. A machine learning algorithm can be used, for example, to determine the physiological parameters and/or symptoms represented by the set of sensor data, and/or whether those parameters and/or symptoms are indicative of the onset of a sleep apnea event. For instance, detection of the onset of a sleep apnea event can be performed based on the output of the machine learning algorithm as well as other patient-specific criteria (e.g., patient-specific changes in physiological parameters such as heart rate, breathing rate, etc.).

If a sleep apnea event is detected (step 420), the control system sends signals to provide simulation therapy to the hypoglossal nerve or geniohyoid muscle in response to the detected sleep apnea event (step 430). The skilled artisan will appreciate that the sensing component 50 may continuously provide physiological data to the control system such that the cessation of a sleep apnea event may be detected. The skilled artisan will also appreciate that steps 410 through 430 may be repeated any number of times, as illustrated by the dashed lined in FIG. 7 (step 440).

In some embodiments, the stimulation frequency, amplitude and pulse duration should be great enough to produce tetanic contraction of one of the muscles innervated by the hypoglossal nerve. In some embodiments, the modulating electric signals have a stimulation frequency of about 10 to about 40 pps. In some embodiments, the modulating electric signals are of an intensity from about 10 to about 3000 microamps (μA). In some embodiments, stimulation amplitudes of up to about 10V, about 15V, about 20 V, about 30 V, about 40V, about 50V, or more can be used for stimulation. In some embodiments, a pulse duration may range from about 0.2 to about 1.0 msec. In other embodiments, the modulating electric signals have a stimulation pulse width of about 10 to about 1000 microseconds (μs). In some embodiments, a frequency applied ranges from between about 25 Hz and about 100 Hz. In some embodiments, the frequency applied ranges from between about 50 Hz and about 100 Hz. In some embodiments, stimulation can begin, for example, about 0.5 seconds, about 1 second, about 5 seconds, more seconds after apnea onset. Of course, the skilled artisan will appreciate that the amount, type, and duration of the stimulation pulse may be adapted depending on the severity of the detected sleep apnea event, or may be adjusted in response to other vital sign measurements obtained from the patient after stimulation of the hypoglossal nerve. Likewise, the above parameters may be adjusted so as to affect stimulation of a patient's geniohyoid muscle.

Charging Device

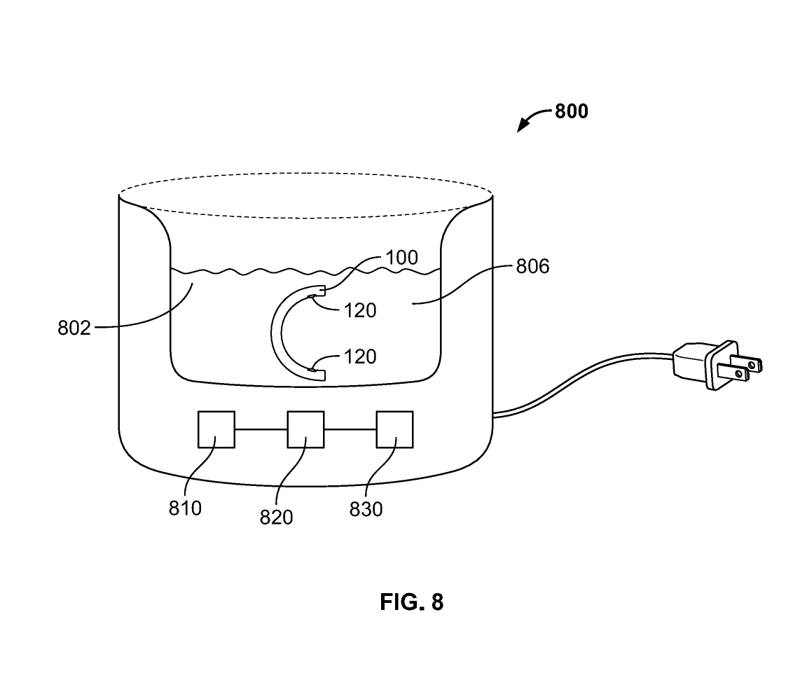

FIG. 8 illustrates an example of an inductively coupled charger device 800 to recharge the stimulation component 100 or the sensing component 50, if so configured. The charger device can include an inductively coupled charging circuit (also referred to as charging circuit) that supplies regulated DC power. The charging circuit 810 can include a transformer 820 to wirelessly transmit power and can include a transmitter controller 830 to control voltage supplied to the transformer.

In some embodiments, the charging device 800 includes a well 806 into which is placed a cleaning solution 802, which is preferably a pleasant-tasting solution that can be used as a mild solvent and antiseptic to clean and sterilize the external surface of the dental stimulator component 100. As noted above, the charging device 800 may inductively charge the power supply of the stimulation component 100 when the stimulator component 100 is disposed in the well 806. Hence, during the day, when the dental stimulator component 800 is not needed, it can be placed in the well 806 for both cleaning and recharging for use later that night.

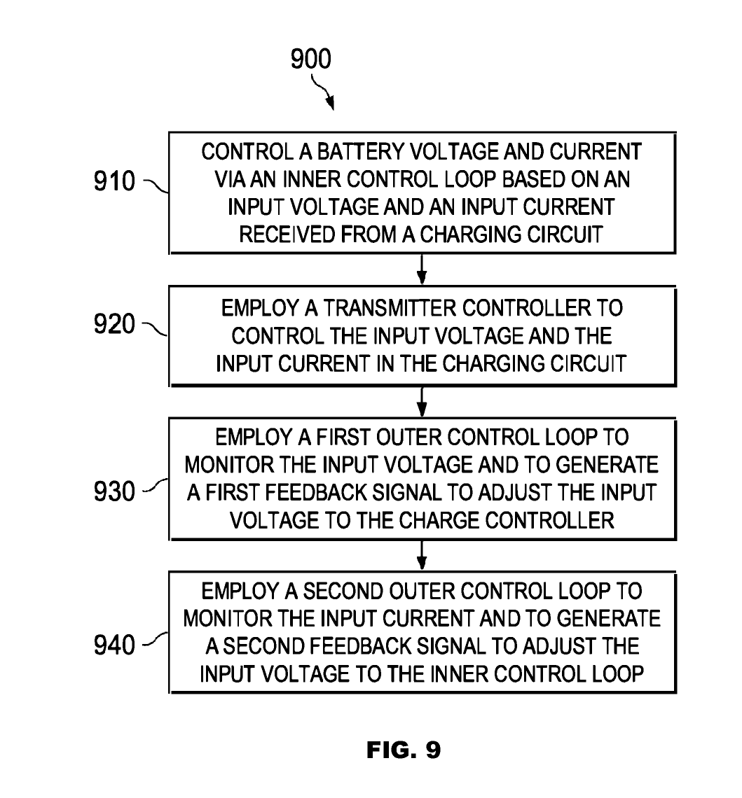

FIG. 9 illustrates an example method for charging a battery of the stimulator component 100 or the sensing component 50 via inductively coupled charging. Proceeding to 910, the method 900 includes controlling a battery voltage and current via an inner control loop based on an input voltage and an input current received from a charging circuit at 910. At 920, the method 900 includes employing a transmitter controller to control the input voltage and the input current in the charging circuit. At 930, the method 900 includes employing a first outer control loop to monitor the input voltage and to generate a first feedback signal to adjust the input voltage to the charge controller. At 940, the method 500 includes employing a second outer control to monitor the input current and to generate a second feedback signal to adjust the input voltage to the inner control loop. The transmitter controller can utilize a PID loop to control the input voltage and the input current in the charging circuit, for example. The method 900 can also include utilizing a regulation switch to control the battery voltage. Additional embodiments for inductively charging a battery, such as the battery within the wearable device described herein, are provided in United States Patent Application Publication No. 2016/0301244, the disclosures of which are hereby incorporated by reference herein in its entirety.

Additional Methods

In some embodiments, the systems described herein may be used to treat facial nerve paralysis. Facial nerve paralysis is a common problem that involves the paralysis of any structures innervated by the facial nerve. Facial nerve paralysis is typically characterized by unilateral facial weakness, with other symptoms including loss of taste, hyperacusis, and decreased salivation and tear secretion. Other signs may be linked to the cause of the paralysis, such as vesicles in the ear, which is may occur if the facial palsy is due to shingles. Conventional facial nerve paralysis treatment options include direct coaptation, interposition nerve grafting, cross-face nerve grafting, and microneurovascular free tissue transfer.

The systems described here may be adapted for use in alleviating the symptoms associated with facial nerve paralysis without the significant surgery associated with the prior art treatments. In some embodiments, a dermal stimulator component 110 can be placed below the paralyzed nerve ending and used to stimulate the muscle when the corresponding muscle is moved on the other side. For example, if a person has a right facial paralysis, a wireless sensor can be placed on the left side muscle and be used to activate the dermal stimulator 110 which is placed on the right side to allow the muscles to move in tandem.

In other embodiments, the systems described herein may be adapted for the treatment of ptosis. Ptosis is a drooping or falling of the upper or lower eyelid. If severe enough and left untreated, the drooping eyelid can cause other conditions, such as amblyopia or astigmatism. Ptosis can be caused by the aponeurosis of the levator muscle, nerve abnormalities, trauma, inflammation or lesions of the lid or orbit. Ptosis may be due to a myogenic, neurogenic, aponeurotic, mechanical or traumatic cause. Conventionally, treatment of ptosis depends on the type of ptosis, and surgical procedures include levator resection, Muller muscle resection, and Frontalis sling operation. Various embodiments can help alleviate the symptoms associated with ptosis without the significant surgery associated with these prior art treatments. Similar to the above discussed embodiments, a dermal stimulator component 110 can be placed in the area of the drooping eyelid muscle and used to stimulate such muscle when the corresponding muscle is moved on the other side. For example, if a person has a drooping right eye, a wireless sensor can be placed near the left eye lid muscle and be used to activate the dermal stimulator 110 which is placed near the right eye lid muscle so that both eyes can be opened and closed together.

Other Components for Practicing Embodiments of the Present Disclosure

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include any number of clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Machine learning algorithms described herein can comprise support vector machines (SVMs). In some instances the SVM provides a linear classification that separates physiological data points having N dimensions into classes based on distance of the data points from a hyperplane having N-1 dimensions. The hyperplane can be chosen so that the distances from the hyperplane to the nearest data points on either side of the hyperplane are maximized, and points lying on opposite sides of the hyperplane are grouped as belonging to distinct classes. In some aspects, points lying on opposite sides of the hyperplane are grouped as belonging to distinct classes corresponding to a “high risk” state versus a “low risk” state for onset of a sleep apnea event. In some aspects the SVM uses a soft margin method for choosing the hyperplane.

In some embodiments, the SVM provides a nonlinear classification that separates the data points with a hyperplane in a transformed feature space. The transformed feature space can be determined by one or more kernel functions, including nonlinear kernel functions. The transformation can be nonlinear and the transformed space high dimensional, such that the classifier can be a hyperplane in the high-dimensional feature space, but can be nonlinear in the original input space. The kernel functions can comprise, without limitation, homogeneous polynomial functions, inhomogeneous polynomial functions, Gaussian radial basis functions, hyperbolic tangent functions, and/or variants thereof and/or combinations thereof.

In some embodiments, the SVM is a multiclass SVM that separates data points into more than two classes. In some embodiment, the multiclass SVM reduces the multiclass problem into multiple binary classification problems. In some embodiments, the multiclass SVM is a directed acyclic graph SVM or a variant thereof In some embodiments, the multiclass SVM uses error-corrected output codes.

Machine learning algorithms described herein can comprise relevance vector machines (RVMs). RVMs can be of similar functional form as SVMs described herein, but can provide probabilistic classifications, such as classifications based on Bayesian inference.

Machine learning algorithms described herein can comprise clustering methods, including but not limited to balanced iterative reducing and clustering using hierarchies (BIRCH). BIRCH can be used to incrementally and dynamically cluster incoming, multi-dimensional physiological data from a patient and to cluster the data optimally for given set of constraints, such as processing constraints, memory constraints and/or speed constraints.

Machine learning algorithms described herein can comprise hierarchical clustering, or hierarchical cluster analysis, that can be used to build a hierarchy of clusters of physiological data. In some embodiments, the hierarchical clustering implements an agglomerative or “bottom up” approach wherein each data point starts in its own cluster, and pairs of clusters are merged at progressively higher levels of the hierarchy. In some embodiments, the hierarchical clustering implements a divisive or “top down” approach wherein all data points start in one cluster, and clusters are split at progressively lower levels of the hierarchy.

Machine learning algorithms described herein can comprise k-means clustering that can be used to physiological data into k clusters, where k is an integer equal or greater than two. After k-means clustering each data point belongs to a cluster having a mean that is closer to the data point than any of the other clusters' means are.

Machine learning algorithms described herein can comprise expectation-maximization (EM) clustering that can be used to determine a maximum likelihood estimate of unobserved latent variables (e.g. unknown physiological parameters) based on a marginal likelihood derived from observed physiological data.

Machine learning algorithms described herein can comprise density-based clustering, such as density-based clustering with noise (DBSCAN) and/or ordering points to identify the clustering structure (OPTICS). Density-based clustering can be used to group together physiological data points that are close to one another and identify data points that are far away from other data points as outliers.

Machine learning algorithms described herein can comprise mean-shift analysis that can be used to determine the maxima of a density function based on discrete physiological data sampled from that function. In some aspects mean-shift analysis can be used to determine one or more maxima corresponding to local or global maxima of density in a plurality of data points lying in a coordinate system for purpose of clustering.

Machine learning algorithms described herein can comprise methods of dimensionality reduction, including but not limited to factor analysis, canonical correlation analysis, principal component analysis, independent component analysis, linear discriminant analysis, Fischer's linear discriminant analysis, non-negative matrix factorization/approximation, t-distributed stochastic neighbor embedding, and/or variants thereof and/or combinations thereof.

Machine learning algorithms described herein can comprise structured prediction and/or structured learning techniques that can be used to predict structured objects and/or structured data, such as structured physiological data. Structured objects and structured data may not be simple data types such as discrete scalar values or real scalar values. Structured objects and structured data may be more complex than simple data types such as discrete scalar values or real scalar values. Structured prediction and/or structured learning techniques can comprise, without limitation, sequence labeling, parsing, collective classification, bipartite matching, graphical models, probabilistic graphical models, Bayesian networks, belief networks, Bayesian models, probabilistic directed acyclic graphical models, conditional random fields, hidden Markov models and/or variants thereof, and/or combinations thereof.

Machine learning algorithms described herein can comprise anomaly detection and/or outlier detection that can be used to identify physiological data that do not conform to an expected pattern or are otherwise distinct from other physiological data in a dataset. Anomaly detection and/or outlier detection can comprise, without limitation, density-based techniques, k-nearest neighbors classification, local outlier factor analysis, subspace-based outlier detection, correlation-based outlier detection, support vector machines, replicator neural networks, cluster analysis, deviations from association rules, deviations from frequent item sets, fuzzy logic based outlier detection, ensemble techniques, feature bagging, score normalization, and/or variants thereof and/or combinations thereof.

Machine learning algorithms described herein can comprise neural networks that can be used to estimate or approximate functions that depend on inputs. The neural networks can comprise one or more layers of artificial “neurons” that receive input data and generate output data. The neural networks can comprise feed-forward and/or feed-back connectivity between “neurons” and/or layers thereof. In some embodiments, the inputs comprise a large number of inputs. The inputs and outputs can comprise physiological data and/or functions thereof. In some aspects the functions are unknown. Neural networks can comprise, without limitation, autoencoder networks, autoassociator networks, Diablo networks, deep learning networks, deep structured learning networks hierarchical learning networks, feedforward artificial neural network models, multilayer perceptrons, recurrent neural networks, In some instances, restricted Boltzmann machines, self-organizing maps, or self-organizing feature maps, convolutional neural networks, and/or variants thereof and/or combinations thereof.

Machine learning algorithms described herein can comprise deep learning methods including but not limited to deep belief networks, deep belief networks, convolutional neural networks, convolutional deep belief networks, deep Boltzmann machines, stacked (denoising) auto-encoders, deep stacking networks, tensor deep stacking networks, Gaussian restricted Boltzmann machines, spike-and-slab restricted Boltzmann machines, compound hierarchical-deep models, deep coding networks, deep kernel machines, deep Q-networks, and/or variants thereof and/or combinations thereof.

Machine learning algorithms described herein can comprise ensemble learning methods that incorporate a plurality of the machine learning methods described herein to obtain better predictive performance than can be achieved from any one of the machine learning methods described herein. The ensemble learning methods can comprise, without limitation, Bayes optimal classifiers, bootstrap aggregating (“bagging”), boosting, Bayesian model averaging, Bayesian model combination, cross-validation selection (“bucket of models”), stacking (stacked generalization), and random forests. In some embodiments, the ensemble learning method comprises random forests that operate by constructing a plurality of decision trees and outputting the class that is the mode of the classes (classification) or mean prediction (regression) of the individual trees.

In alternative embodiments, the systems and methods described herein may not use a machine learning algorithm to perform the patient-customized monitoring and treatment of the present disclosure. In some embodiments, the systems and methods described herein may use an algorithm, process and/or method that is not a machine learning algorithm instead of or in addition to a machine learning algorithm to perform the patient-customized monitoring and treatment of the present disclosure.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A system for the treatment of obstructive sleep apnea in a patient in need thereof, the system comprising a sensing component and a stimulation component, the sensing component comprising one or more wireless sensors for collecting one or more vital signs of the patient, the sensing component being in wireless communication with a control system, and wherein the stimulation component comprises (i) a surgically implantable body configured to deliver energy to one of a nerve or muscle; and (ii) a wearable appliance inductively coupled to the implanted body, the wearable portion configured to receive signals from the control system.

2. The system of claim 2, where the vital signs are selected form the group consisting of blood oxygen, respiration rate, and heart rate.

3. The system of claim 1, wherein the wearable portion is a dental appliance comprising a rechargeable battery, a pulse generator, and a means for inductively delivering energy to the surgically implantable body.

4. The system of claim 3, wherein the means for inductively delivering energy is a transmitter coil.

5. The system of claim 4, wherein the surgically implantable body comprises a receiver coil for receiving energy from the wearable portion.

6. The system of claim 3, wherein the surgically implantable body is configured to deliver energy to a hypoglossal nerve.

7. The system of claim 1, wherein the wearable portion is a dermal device for positioning on the patient's skin, and wherein the dermal device comprises a means for inductively delivering energy to the surgically implantable body.

8. The system of claim 7, wherein the surgically implantable body is configured to deliver energy to a geniohyoid muscle.

9. The system of claim 8, wherein the surgically implantable body comprises a means for wirelessly receiving energy from the dermal device, and wherein the surgically implantable device further comprises an insulating disc.

10. The system of claim 1, wherein the control system is embedded within the wearable apparatus of the stimulation component.

11. An apparatus for treating sleep apnea comprising: an implantable body having a first member of a pair of inductive power transfer coils; and a wearable apparatus having a second member of the pair of inductive power transfer coils, a rechargeable battery, and a pulse generator; wherein the wearable apparatus is configured to wirelessly deliver energy to the implantable body upon receipt of a signal indicative of a sleep apnea event; and wherein the implantable body is configured to transfer the energy received from the wearable apparatus to a hypoglossal nerve or a geniohyoid muscle positioned in proximity thereto.

12. The apparatus of claim 11, wherein the wearable apparatus is a dental appliance adapted for placement over the patient's lower teeth.

13. The apparatus of claim 12, wherein the dental appliance is a bitesplint or retainer.

14. The apparatus of claim 11, wherein the wearable apparatus further comprises means for receiving control signals from a control system communicatively coupled thereto.

15. The apparatus of claim 11, wherein the control system comprises a processor, a memory, and a wireless communications module, the control system configured to (i) receive signals from one or more wireless sensors, (ii) process the signals to determine if a sleep apnea event has occurred or will occur, and (iii) send control signals to the wearable apparatus.

16. The apparatus of claim 11, wherein the control system is embedded within the dental appliance.

17. A system for treating sleep apnea in a patient in need of treatment thereof comprising (i) one or more wireless sensors, (ii) a stimulation device, the stimulation device having a wearable portion and an implantable portion, the wearable portion configured to wirelessly transmit stimulation pulses to the implantable portion, and (iii) a control system, the control system having a memory coupled to the one or more processors, the memory to store computer-executable instructions that, when executed by the one or more processors, cause the one or more processors to perform operations comprising (a) measuring vital signs of a patient using the one or more wireless sensors; (b) determining whether a sleep apnea event has occurred or will occur based on the measured vital signs; and (c) facilitating the delivery of a stimulation pulse to treat sleep apnea using the stimulation component; wherein the control system is in wireless communication with both the one or more wireless sensors and the wearable portion of the stimulation component.

18. The system of claim 17, wherein the measured vital signs are used to derive a sleep apnea index, and wherein the step of determining whether the sleep apnea event has occurred or will occur comprises comparing the derived sleep apnea index to a pre-determined sleep apnea index specific for the patient, the pre-determined sleep apnea index being stored in the memory.

19. The system of claim 17, wherein the one or more wireless sensors include a respiration sensor, and wherein the step of determining whether the sleep apnea event has occurred or will occur comprises comparing measured respiration rates to a pre-determined threshold respiration rate.

20. The system of claim 17, wherein the wearable apparatus is a dental appliance, and wherein the dental appliance comprises at least two transmission coils for delivering the stimulation pulses to two implantable bodies, the dental appliance adapted to releasably engage a portion of the patient's lower teeth, the at least two transmission coils positioned on an exterior surface of the dental appliance.
